(19) United States
(12) Patent Application Publication
(10) Pub. No.: US 2003/0153965 A1
(43) Pub. Date: Aug. 14, 2003

(54) Electrically conducting nanocomposite materials for biomedical applications

(75) Inventors: Peter Supronowicz, Trenton, NJ (US); Rena Bizios, Troy, NY (US); Pulickel Ajayan, Clifton Park, NY (US); Richard Siegel, Menands, NY (US)

Correspondence Address: HESLIN ROTHENBERG FARLEY & MESITI PC, 5 COLUMBIA CIRCLE, ALBANY, NY, 12203, US

(73) Assignee: RENSSELAER POLYTECHNIC INSTITUTE, Troy, NY

(21) Appl. No.: 10/298,158

(22) Filed: Nov. 15, 2002

Related U.S. Application Data
(63) Continuation of application No. PCT/US01/15910, filed on May 16, 2001.
(60) Provisional application No. 60/204,416, filed on May 16, 2000.

(51) Int. Cl. A61N 1/05

(52) U.S. Cl. 607/116

(57) ABSTRACT

Exposing osteoblasts on an electrically conducting nanocomposite, which may be an orthopaedic/dental implant, to electrical stimulation enhances osteoblast proliferation thereon. The electrically conducting nanoscale material includes an electrically conducting nanoscale material and a biocompatible polymer and/or a biocompatible ceramic; carbon nanotubes may be used as the electrically conducting nanoscale material.

### BACKGROUND OF THE INVENTION

Electrical stimulation has been explored as a treatment for damaged bone tissue since shortly after the discovery in the late 1950's of the presence of electrical potentials in mechanically loaded bone. Various animal models have provided evidence that electrical stimulation enhances bone healing. For example, increased new bone formation was reported when electric currents of 5-20 μA were applied continuously to osteotomies in animal models for 14 days [Friedenburg et al. “Bone Reaction to Varying Amounts of Direct Current” Gynecological Obstetrics 131, 894-899 (1970)]; however, the mechanisms behind these events are still not fully understood.

Typically, electric current (such as the direct current electrical stimulation used in animal studies) has been delivered to bone through metal (specifically, stainless steel, platinum, and titanium) electrodes. At the end of the treatment process, after bone repair had occurred, the implanted metal electrodes were removed from the site of newly healed bone tissue via a surgical procedure. Risk of complications of the surgery, such as infection at the site of implantation and damage to the newly formed bone tissue (especially when the metal electrode had integrated and/or bonded to the apposing bone tissue) is a major disadvantage of this approach. A second disadvantage is the limited extent to which the electrical stimulus could be delivered to damaged bone; new bone formation occurred only near the electrode tip and did not encompass the extent of the damaged and/or fractured bone tissue.

In addition to implanted metal electrodes, some isolated attempts for delivering electrical stimulation to cultured cells and to animal extremities have been made; however, due to (at best) partial success, these methodologies were neither pursued further nor widely implemented. Capacitively coupled electric fields, while suitable for delivering electrical current to cultured cells, have had limited use in larger animal models due to the high (in excess of 1,000 volts) voltages that accompany the increase in plate gap distance required to accommodate the limbs of larger animals. Conversely, direct current electrical stimulation, while adequate for in vivo applications, has shortcomings in vitro arising from accumulation of charged chemical compounds (contained within the supernatant media) on the electrodes used to expose cultured cells to the electrical current; build-up of proteins on the electrodes leads to decreases in the magnitude of the electrical stimulus and, consequently, limits the effectiveness of this method for bone healing purposes.

For these reasons, use of electrical stimulation to treat bone fractures in clinical applications has been limited. There is, therefore, a need for methodologies utilizing new current-conducting material formulations.

Careful design of biomaterials is important to improve biomedical implant success rates and biorepair capability. The materials for these implants require special properties that enhance their biocompatibility (specifically, attachment, proliferation and specialized functions of cells), and that also exhibit and/or enhance their desirable mechanical and biophysical (such as electrical, piezoelectric, and magnetic) properties. There is, therefore, a need for new biomaterials that improve cytocompatibility and improve specific cell functions.

### SUMMARY OF THE INVENTION

It has been unexpectedly discovered that electrically conducting nanocomposites according to the present invention can improve cytocompatibility and improve specific cell functions. A nanoscale material is defined herein as any material having at least one dimension in the nanoscale range. The nanoscale range begins at about the diameter of an atom, which is generally greater than 0.1 nm, and ends at about 100 nm. Preferably, the nanoscale range begins at about 0.5-1 nm.

Accordingly, the present invention relates to an electrically conducting nanocomposite that includes an electrically conducting nanoscale material and a biocompatible polymer and/or a biocompatible ceramic. The electrically conducting nanoscale material may be a carbon nanotube, an inorganic nanotube, a metal nanowire, a ceramic nanowire, a composite nanowire, a metal nanofilament, a ceramic nanofilament, a composite nanofilament or a combination thereof; in particular, it may be a carbon nanotube. Where the electrically conducting nanocomposite includes a nanoscale electrically conducting material and a biocompatible polymer, the polymer may be biodegradable or nonbiodegradable. In some cases, a preferred biocompatible polymer is biodegradable; in particular, the polymer may be polylactic acid. A useful electrically conducting nanocomposite includes carbon nanotubes and polylactic acid. Where the electrically conducting nanocomposite includes a nanoscale electrically conducting material and a biocompatible ceramic, the ceramic may have a grain size of 1-100 nm. In particular, the ceramic may be alumina, titania or hydroxyapatite.

In another aspect, the invention relates to a method for enhancing osteoblast proliferation on a surface of 2-dimensional substrate or inside a 3-dimensional scaffold of an electrically conducting orthopaedic/dental implant. The method includes contacting the implant with osteoblasts, and passing an electric current through the implant; whereby the osteoblasts are exposed to electrical stimulation. In particular, the electric current may be an alternating current.

### DETAILED DESCRIPTION OF THE INVENTION

An electrically conducting nanocomposite according to the present invention comprises an electrically conducting nanoscale material, and at least one of a biocompatible polymer or a biocompatible ceramic. The electrically conducting nanoscale material may be a carbon nanotube, an inorganic nanotube, a metal nanowire, a ceramic nanowire, a composite nanowire, a metal nanofilament, a ceramic nanofilament, a composite nanofilament or a combination thereof. In particular, the electrically conducting nanoscale material may be a carbon nanotube. The biocompatible polymer may be any cytocompatible, or biocompatible polymer. It is preferably bioabsorbable and/or bioerodable, and is also non-toxic, noncrcinogenic, and causes no adverse immunologic response. Representative useful materials include: polyfumarates; polylactides; polyglycolides; polycaprolactones; polyanhydrides; pyrollidones, for example, methylpyrollidone; cellulosic polymers; for example, carboxymethyl cellulose; methacrylates; collagens, for example, gelatin; glycerin and polylactic acid. Synthetic polymer resins may also be used, including, for example, epoxy resins, polycarbonates, silicones, polyesters, polyethers, polyolefins, synthetic rubbers, polyurethanes, nylons, polyvinylaromatics, acrylics, polyamides, polyimides, phenolics, polyvinylhalides, polyphenylene oxide, polyketones and copolymers and blends thereof. Copolymers include both random and block copolymers. Polyolefin resins include polybutylene, polypropylene and polyethylene, such as low density polyethylene, medium density polyethylene, high density polyethylene, and ethylene copolymers; polyvinylhalide resins include polyvinyl chloride polymers and copolymers and polyvinylidene chloride polymers and copolymers, fluoropolymers; polyvinylaromatic resins include polystyrene polymers and copolymers and poly α-methylstyrene polymers and copolymers; acrylate resins include polymers and copolymers of acrylate and methacrylate esters, polyamide resins include nylon 6, nylon 11, and nylon 12, as well as polyamide copolymers and blends thereof; polyester resins include polyalkylene terephthalates, such as polyethylene terephthalate and polybutylene terephthalate, as well as polyester copolymers; synthetic rubbers include styrenebutadiene and acrylonitrilebutadiene-styrene copolymers; polyketones include polyetherketones and polyetheretherketones. The polymer is preferably polylactic acid. The biocompatible polymer may be a biodegradable polymer. Suitable biodegradable polymers include, for example, polyglycolide (PGA), including polyglycolic acid, copolymers of glycolide, glycolide/L-lactide copolymers (PGA/PLLA), lactide/trimethylene carbonate copolymers (PLA/TMC), glycolide/trimethylene carbonate copolymers (PGA/TMC), polylactides (PLA), including polylactic acid, stereo-copolymers of PLA, poly-L-lactide (PLLA), poly-DL-lactide (PDLLA), L-lactide/DL-lactide copolymers, copolymers of PLA, lactide/tetramethylglycolide copolymers, lactide/α-valerolactone copolymers, lactide/ε-caprolactone copolymers, hyaluronic acid and its derivatives, polydepsipeptides, PLApolyethylene oxide copolymers, unsymmetrical 3,6-substituted poly-1,4-dioxane-2,5-diones, poly-β-hydroxybutyrate (PHBA), HBA/β-hydroxyvalerate copolymers (PHBA/HVA), poly-p-dioxanone (PDS), poly-α-valerolactone, poly-ε-caprolactone, methacrylate-N-vinyl-pyrrolidone copolymers, polyesteramides, polyesters of oxalic acid, polydihydropyranes, polyalkyl-2-cyanoacrylates, polyurethanes, polyvinylalcohol, polypeptides, poly-B-malic acid (PMLA), poly-B-alcanoic acids, polybutylene oxalate, polyethylene adipate, polyethylene carbonate, polybutylene carbonate, and other polyesters containing silyl ethers, acetals, or ketals, alginates, and blends or other combinations of the aforementioned polymers. In addition to the aforementioned aliphatic link polymers, other aliphatic polyesters may also be appropriate for producing aromatictaliphatic polyester copolymers. These include aliphatic polyesters selected from the group of oxalates, malonates, succinates, glutarates, adipates, pimelates, suberates, azelates, sebacates, nonanedioates, glycolates, and mixtures thereof. These materials are useful as biodegradable support membranes in applications requiring temporary support during tissue or organ regeneration. In particular polylactic acid may be used in the composite of the biocompatible polymer and the electrically conducting nanoscale material.

The biocompatible ceramic may be any biocompatible ceramic, including oxides, nitrides, borides and carbides of silicon, zirconium, aluminum, magnesium, and yttrium; complex ceramic compounds such as SiAION. Examples of such ceramic compositions are silicon nitride, silicon carbide, zirconia, alumina, titania, mullite, silica, a spinel, SiAION, and mixtures thereof. In particular, the biocompatible ceramic may be hydroxyapatite, alumina or titania. The biocompatible ceramic may be a nanoscale material in its own right, having a grain size ranging from 1 to 100 nm.

The amount of electrically conducting nanoscale material in the composite should be sufficiently high to impart electrical conductivity to the composite. Typically, conductivity requires a contiguous, or nearly contiguous, arrangement of the nanotubes, nanofilaments, or nanowires. In particular, the electrically conducting nanoscale material may form an interpenetrating network within a matrix of the biocompatible polymer or the biocompatible ceramic. The amount of electrically conducting nanoscale material then, ranges from 0.1 to 90 parts per volume, and the amount of the biocompatible polymer or the biocompatible ceramic ranges from 10 to 99.9 parts per volume. In particular, the amount of the electrically conducting nanoscale material may range from about 10 to 25 parts by volume and the amount of the biocompatible polymer or biocompatible ceramic may range from about 75 to about 90 parts per volume. In one embodiment an electrically conducting nanocomposite according to the present invention comprises a carbon nanotube and polylactic acid. In this nanocomposite, the amount of the carbon nanotubes may range from about 20 to 25 parts by weight, and the amount of the polylactic acid may range from about 70 to 80 parts by weight.

In another embodiment, the present invention relates to an electrically conducting nanocomposite comprising a nanoscale material and at least one of a biocompatible polymer or a biocompatible ceramic; at least one of the nanoscale material, polymer and ceramic is electrically conducting. Electrically conducting nanoscale materials are described above. Electrically conducting polymers and ceramics are known, and will not be further described here.

In yet another embodiment, the present invention relates to a method for enhancing osteoblast proliferation on the surface of an 2-dimensional substrate or a 3-dimensional scaffold of an electrically conducting orthopaedic/dental implant. The method includes contacting the implant with osteoblasts, and passing an electric current through the implant. By this method, the osteoblasts are exposed to electrical stimulation. The electric current may be generated by a pulse/function generator through direct contact with the implant, or induced therein by an pulsed electromagnetic field. The implant may be temporary, short-term or long-term. In addition, bone repair in the area where the osteoblasts are exposed to electrical stimulation may be improved.

The electrically conducting nanocomposite of the present invention may be used as an in vitro or in vivo tissue engineering scaffold or substrate. Such a substrate or scaffold may be 2- or 3-dimensional, and porous or non-porous. Bony material may be generated on a scaffold under electrical stimulation. This material may used for tissue repair, for example, as a bone filler. An electrically conducting nanocomposite may also be used as part of a system for providing controlled electrical stimulation to a cell, tissue, organ or body part of a human being or an animal. In particular, it may be used as an in vitro or in vivo biosensor for use in a diagnostic procedure. The electrically conducting nanocomposite may also be used in vitro or in vivo for probing, substituting for, repairing or regenerating a cell, tissue, organ, or body part of any human being or an animal. The tissue may be central or peripheral nerve tissue, or it may be bone tissue.

The electrically conductive nanocomposite may additionally comprise a filler. The filler may be a pigment, an inorganic solid, a metal, or an organic. Typical pigments include: titanium dioxide, carbon black, and graphite. Other inorganic fillers include talc, calcium carbonate, silica, aluminum oxide, glass spheres (hollow or solid) of various particle sizes, nanometer-sized particles of silica or alumina, mica, corundum, wollastonite, silicon nitride, boron nitride, aluminum nitride, silicon carbide, beryllia, and clays. Metallic fillers include copper, aluminum, stainless steel and iron. Organic fillers include wax and crosslinked rubber particles. Fillers may be chosen based on cost, thermal properties, and mechanical properties desired. Particle size of the filler may range from the nanoscale range, to 0.01 to 100 microns.

### EXAMPLES

### Example 1

### Polylactic Acid (PLA)/Carbon Nanotube (CNT) Composites

Multi-walled carbon nanotubes (0.1 gm) produced using the electric arc method [Ajayan “Nanotubes from Carbon” Chemical Reviews 99, 1787-1799 (1999)] were added to an emulsion of PLA (molecular weight 100,000) pellets (0.35 gm) in 4 mL of chloroform. The polymer/carbon nanotube slurry was then sonicated for 15 minutes and air-dried for 48 hours. To ensure full evaporation of the solvent, each PLA/CNT composite was vacuum-dried at room temperature for 24 hours, heated to 130° C., and allowed to cool at room temperature. This process yielded non-porous PLA/CNT disks (each 40 mm in diameter and 1 mm thick).

Representative cross-sections of PLA/CNT composites were sputter-coated with gold and examined by scanning electron microscopy (JEOL JSM T-300) using standard procedures [Squire et al. “Analysis of Osteoblast Mineral Deposits on Orthopaedic/Dental Implant Metals” Biomaterials 17, 725-733 (1996)]. Micrographs of representative sample cross-sections were taken from the perspective of fracture surfaces. In addition, the electrical resistance of the PLA/CNT composites was determined using a universal probe (Jandel Engineering) and following manufacturer's instructions.

The surfaces of the planar PLA/CNT composites used in the present study were found to be homogeneous, smooth, and non-porous. Carbon nanotubes were distributed throughout the polymer phase of the composite substrate.

The electrical resistance of the substrates used in the present study was measured using a three-point probe. Polylactic acid is an insulator and does not conduct electricity. In contrast, the 80/20% (w/w) PLA/CNT composite tested in the present study was a conductive material with a finite resistance of 200 ohms.

### Examples 24

### Experimental Procedure

Cell Culture

Osteoblasts were isolated via sequential collagenase digestions of Sprague-Dawley rat calvaria according to established techniques [Puleo et al. “Osteoblast Responses to Orthopedic Implants” J. Biomed. Mat. Res. 25, 725-733 (1996) and were cultured in Dulbecco's Modified Eagle Medium (DMEM), supplemented with 10% fetal bovine serum, under standard cell culture conditions (i.e., a sterile, 37° C., humidified, 5% CO₂/95% air environment). The osteoblastic phenotype of these cells was determined by expression of genes for alkaline phosphatase, osteopontin, osteonectin, osteocalcin, and collagen type I as well as by the presence of calcium mineral in the extracellular matrix.

Osteoblasts passage number 2-3 were used in the experiments of the present study.

Alternating Electric Current System

In order to culture cells on the surface of each PLA/CNT-composite substrate, a special housing was constructed to hold the necessary cell-culture media and to maintain sterile conditions. Individual hollow polypropylene cylinders (1.5 cm in diameter, 3 cm long, Fisher) were glued onto the top surface of each PLA/CNT composite substrate using a bead of silicone glue along the outside perimeter of each tube. These wells were sterilized in 70% ethanol for 20 minutes and were rinsed in sterile PBS for 5 minutes prior to use in cell experiments.

Osteoblasts were exposed to electric current stimulation using a custom-built laboratory system. In this system, a stainless steel electrode was immersed into the supernatant media at a distance of 0.5 cm from cells cultured onto the surface of individual current conducting PLA/CNT composite substrates. Alternately, the electric current was passed through the PLA/CNT composite substrate. An HP8110A pulse/function generator provided the electrical stimulus, consisting of an alternating current of 10 μA at a frequency of 10 Hz with a 50% duty cycle.

### Example 2

### Osteoblast Proliferation

Osteoblasts suspended in DMEM (containing 10% fetal bovine serum) were seeded sub-confluently at a density of 2,500 cells per square centimeter of PLA/CNT composite substrate surface area and allowed to adhere in a sterile, 37° C., humidified, 5% CO₂/95% air environment for 24 hours. The cells were then exposed to electrical stimulation (10 μA at 10 Hz) for 6 hours daily for 2 consecutive days. Controls were osteoblast proliferation experiments run in parallel and maintained under similar cell culture conditions, but not exposed to any electrical stimulation.

At the end of the prescribed time period, adherent cells were rinsed with PBS, fixed with 10% formalin, stained with 10⁻⁶ M Hoechst No. 33258, and counted in situ in five random fields per substrate using fluorescent microscopy (365 nm excitation/400 nm emission; Olympus).

The cell proliferation experiments were run in triplicate and repeated at four separate times.

Osteoblast proliferation increased significantly (p<0.03) from 15,810±4,813 (mean±SEM) cells on the PLA/CNT composite substrates under control (no electrical stimulation) conditions to 31,574±7,076 (mean±SEM) cells after exposure to 10 μA at 10 Hz of electrical stimulation for 6 hours daily for 2 consecutive days. This result represents a 46% increase in osteoblast proliferation after exposure to electrical stimulation.

### Example 3

### Calcium-Containing Mineral In the Extracellular Matrix

Osteoblasts suspended in DMEM (supplemented with 10% fetal bovine serum, 50 μg/mL ascorbic acid, and 10 mM β-glycerophosphate) were seeded at a density of 75,000 cells per square centimeter of PLA/CNT-composite substrate surface area. These confluent osteoblasts were cultured in a sterile, 37° C., humidified, 5% CO₂/95% air environment for 48 hours before they were exposed to alternating current stimulation for 6 hours daily for 21 consecutive days. Controls were osteoblast maintained under similar cell culture conditions, but not exposed to any electrical stimulation. Supernatant media in all samples were changed every 4 days for the duration of the experiments.

At the end of the 21-day time period, cell cultures were rinsed twice with calcium-free/magnesium-free PBS and lysed with 0.5 N HCI by shaking at 4° C. for 6 hours. After centrifugation (500×g for 5 minutes), the calcium concentration in the supernatants was determined using Calcium Kit #587 (Sigma) and following manufacturer's instructions. Light absorbance of the calcium-containing samples was determined spectrophotometrically (575 nm). Total calcium (mg/dL) was calculated from standard curves of absorbance versus known concentrations (specifically, 5, 10, and 15 mg/dL) of calcium samples (assayed in parallel with samples from both osteoblasts exposed to electrical stimulation and those maintained under control, that is, no electrical stimulation conditions). The experiments to quantify calcium concentration in the extracellular matrix were run in triplicate and repeated at three separate times.

Compared to 45±9 (mean±SEM) μg calcium that was synthesized and deposited in the extracellular matrix by osteoblasts cultured on the PLA/CNT composite substrates under control (no electrical stimulation) conditions, the amount of calcium increased significantly (p<0.01) to 138±19 (mean±SEM) μg following osteoblast exposure to 10 μA at 10 Hz of electrical stimulation for 6 hours daily for 21 consecutive days; this result represents a 307% increase in calcium content.

### Example 4

### Reverse Transcription-Polymerase Chain Reaction for Semiquantitation of Select Gene Expression

Osteoblasts suspended in DMEM (supplemented with 10% fetal bovine serum, 50 μg/mL ascorbic acid, and 10 mM β-glycero-phosphate) were seeded onto the surface of PLA/CNT composite samples at a density of 75,000 cells per square centimeter of substrate surface area. These confluent cells were cultured in a sterile, 37° C., humidified, 5% CO₂/95% air environment for 48 hours before they were exposed to alternating current stimulation for 6 hours a day for either 1 or 21 days. Controls were osteoblasts maintained under similar cell culture conditions, without exposure to electrical stimulation.

At the end of the prescribed time periods, the osteoblasts were rinsed twice with calcium free/magnesium free PBS and total cellular RNA was extracted with Trizol Reagent (Life Technologies) using standard procedures. One microgram of total RNA was reverse transcribed into cDNA using a reverse transcription kit (Life Technologies) and oligo (dT) primers according to published techniques. [Arulanandam et al. “Modulation of Mucosal and Systemic Immunity by Intranasal Interleukin 12 Delivery” Vaccine 17, 252-260 (1999)]. After incubation at 25° C. for 10 minutes and at 42° C. for 60 minutes, the resulting cDNA was amplified using specific primers for alkaline phosphatase, osteopontin, osteocalcin, collagen type I, osteonectin, osteoprotegerin, and bone sialoprotein with hypoxanthine phosphoribosyl transferase (HPRT) primers as controls. PCR amplification was performed by processing 2 μL of cDNA with a PCR core kit (Life Technologies) and subjecting the resulting mixture to the following amplification profile: denaturing at 95° C. for 1 minute (for all primers), annealing at 56° C. for 1 minute (for alkaline phosphatase, osteopontin, and HPRT primers) or at 65° C. (for osteocalcin and collagen type I primers), and extension at 72° C. for 1 minute (for all primers) for a duration of 28 cycles. PCR amplification was followed by a final extension at 72° C. for 10 minutes. The PCR products were separated on a 2.5% agarose gel, stained with ethidium bromide, and visualized using UV transillumination.

Exposure of osteoblasts to 10 μA at 10 Hz of electrical stimulation for 6 hours daily for up to 21 consecutive days differentially affected expression of various genes. Specifically, there was no detectable gene expression for either alkaline phosphatase or bone sialoprotein under any condition or time point tested. Compared to controls, osteopontin was slightly down-regulated in cells exposed to 6 hours of electrical stimulation; after 21 consecutive days, however, expression of osteopontin was similar both in controls and in osteoblasts exposed to electrical stimulation. Osteonectin mRNA was expressed when osteoblasts were maintained under control conditions, but not when these cells were exposed to electrical stimulation for 6 hours; in contrast to controls, expression of osteonectin was upregulated in osteoblasts exposed to electrical stimultion for 6 hours daily for 21 consecutive days.

There was no detectable gene expression of osteocalcin in osteoblasts either under control conditions or under electrical stimulation for 6 hours. Compared to controls, however, the gene for osteocalcin was upregulated in cells exposed to electrical stimulation for 6 hours daily for 21 consecutive days. There was no detectable collagen type I expression in osteoblasts maintained under control conditions; in contrast, there was significant expression of the collagen type I gene after both 6 hours and after 21 consecutive days of electrical stimulation for 6 hours daily.

Gene expression for osteoprotegerin was similar in both controls and in cells exposed to electrical stimulation for 6 hours. Gene expression for osteoprotegerin, however, was significantly upregulated when osteoblasts were exposed to electrical stimulation for 21 consecutive days for 6 hours daily.

HPRT, the housekeeping gene, was equally expressed in osteoblasts maintained under control conditions and in osteoblasts exposed to electrical stimulation for 6 hours daily for 1 and 21 consecutive days. HPRT was used for quality assurance purposes to monitor consistency of the technique.

In contrast to polylactic acid (and to most other polymers) which is an insulator, the novel 80/20% (w/w) PLA/CNT composite which was prepared in the present study is a conductive material. Availability of these novel material formulations and of well-characterized cellular models made possible a series of studies on the effect of alternating electric current stimulation at the cellular/molecular level. Since bone repair, healing, and regeneration in humans and animals involve major changes in bone tissue formation, the present study focused on aspects pertinent to new bone formation; for an in vitro model these included osteoblast proliferation as well as synthesis of chemical constituents of the bone matrix.

Evidence that electrical stimulation enhances osteoblast proliferation has been provided in the literature. See, for example, Brighton et al., “In vitro Bone-cell Response to a Capacitively Coupled Electrical Field,” Clin. Ortho. Related. Res. 285, 255-262 (1992). The present study, however, is the first to report 46% increases in proliferation when osteoblasts, cultured on current-conducting PLA/CNT composites, were exposed to alternating electric current stimulation.

Direct comparison of the results of all these studies is not possible because of differences in delivering the electrical stimulus. For example, compared to conditions reported in the literature, the present study utilized electric currents ten times lower in magnitude, but obtained similar increases in cell proliferation. In contrast, studies performed by other researchers exposed osteoblasts and/or osteoblast-like cells to electrical stimulation over shorter periods of time using capacitively coupled electric fields and direct current electrical stimulation.

Production and deposition of calcium-containing mineral, the osteoblast function directly responsible for the inorganic phase of bone (which accounts for approximately 65% of total bone mass) was enhanced threefold in osteoblasts exposed to alternating current electrical stimulation. The increased calcium-containing mineral observed in these in vitro studies might provide an explanation for the accelerated healing observed in several animal models of osteotomies and fractures that underwent treatment using electrical stimulation.

What unequivocally distinguishes the present study from previous reports in the literature is evidence that alternating current electrical stimulation induces molecular responses that affect transcription of genes pertinent to bone-matrix composition and homeostasis. First, and foremost, upregulation of the collagen type I (the major, approximately 90%, constituent of the organic phase of bone) gene was manifested as early as 6 hours and remained upregulated after 21 consecutive days (6 hours daily) of exposure to alternating current electrical stimulation. In addition, genes for two other proteins, specifically osteonectin and osteopontin, which play a role in the mineralization of the extracellular matrix of bone, were also upregulated under the conditions tested in the present study. These results suggest that upregulation of osteonectin, a phosphoprotein which is involved in creating nucleation points for calcium deposition, as well as upregulation of osteocalcin, a γ-carboxyglutamic acid-containing protein which is found exclusively in bone and has been proposed to regulate crystal growth, may be part of the mechanism of extracellular matrix formation and mineralization under alternating current electrical stimulation.

In addition, the present study provided the first molecular-level evidence that alternating current electrical stimulation may affect two osteoblast-produced proteins that have proposed roles in modulating osteoclast functions relevant to bone mineral resorption. Since osteoclast attachment to the extracellular matrix is a prerequisite for their subsequent resorption of calcium-containing mineral, decreased production of osteopontin may have critical implications in inhibiting attachment of osteoclasts to the mineralized extracellular matrix. Moreover, since osteoprotegerin, a member of the tumor necrosis factor family of receptors, inhibits osteoclast differentiation and activation, the observed gene upregulation in osteoblasts indicates another possible mechanism that may control the bone-resorptive activity of osteoclasts. In this respect, the increased bone formation observed in animal models exposed to electrical stimulation may be the result of enhanced select osteoblast functions and concomitant controlled select functions of osteoclasts.

What is claimed:

1. An electrically conducting nanocomposite comprising an electrically conducting nanoscale material and at least one of a biocompatible polymer or a biocompatible ceramic.

2. An electrically conducting nanocomposite according to claim 1 wherein the electrically conducting nanoscale material comprises a carbon nanotube, an inorganic nanotube, a metal nanowire, a ceramic nanowire, a composite nanowire, a metal nanofilament, a ceramic nanofilament, a composite nanofilament and combinations thereof.

3. An electrically conducting nanocomposite according to claim 1 wherein the nanoscale material is a carbon nanotube.

4. An electrically conducting nanocomposite according to claim 1 comprising a nanoscale electrically conducting material and a biocompatible polymer.

5. An electrically conducting nanocomposite according to claim 4, wherein the biocompatible polymer is biodegradable.

6. An electrically conducting nanocomposite according to claim 5, wherein the biocompatible polymer is polylactic acid.

7. An electrically conducting nanocomposite according to claim 1 comprising carbon nanotubes and polylactic acid.

8. An electrically conducting nanocomposite according to claim 1 comprising a nanoscale electrically conducting material and a biocompatible ceramic.

9. An electrically conducting nanocomposite according to claim 8, wherein the ceramic has a grain size of 1-100 nm.

10. An electrically conducting nanocomposite according to claim 8, wherein the ceramic is alumina, titania or hydroxyapatite.

11. An electrically conducting nanocomposite according to claim 1 comprising:
about 0.1-90 parts by volume of an electrically conducting nanoscale material; and
about 10-99.9 parts by volume of at least one of a biocompatible polymer or a biocompatible ceramic.

12. An electrically conducting nanocomposite according to claim 11 comprising:
about 10-25 parts by volume of an electrically conducting nanoscale material ; and
about 75-90 parts by volume of at least one of a biocompatible polymer or a biocompatible ceramic.

13. An electrically conducting nanocomposite according to claim 12, comprising
carbon nanotubes, and
polylactic acid.

14. The electrically conducting nanocomposite according to claim 13 comprising
20-25 parts by weight carbon nanotubes; and
75-80 parts by weight polylactic acid.

15. A method for enhancing osteoblast proliferation on a surface of 2-dimensional substrate or inside a 3-dimension scaffold of an electrically conducting orthopaedic/dental implant, said method comprising:
contacting the implant with osteoblasts; and
passing an electric current through the implant; whereby the osteoblasts are exposed to electrical stimulation.

16. A method according to claim 15, wherein the electric current is produced by a pulse/function generator directly connected to the implant.

17. A method according to claim 15, wherein the electric current is induced in the implant by a pulsed electromagnetic field.

18. A method according to claim 15, wherein the electric current is an alternating current.

19. An electrically conducting nanocomposite comprising a nanoscale material and at least one of a biocompatible polymer or a biocompatible ceramic, wherein at least one of said nanoscale material, said polymer and said ceramic is electrically conducting.

20. An electrically conducting nanocomposite according to claim 19, wherein the nanoscale material is electrically conducting.

21. An electrically conducting nanocomposite according to claim 19, wherein the biocompatible polymer is electrically conducting.

22. An electrically conducting nanocomposite according to claim 19, wherein the biocompatible ceramic is electrically conducting.
